(19) United States
(12) Patent Application Publication
(10) Pub. No.: US 2026/0115412 A1
(43) Pub. Date: Apr. 30, 2026

(54) CATHETER ASSEMBLY AND RELATED METHODS

(71) Applicant: Excision Medical, Inc., Exton, PA (US)

(72) Inventors: David Alexander WOOD, Vancouver (CA); Janarthanan SATHANANTHAN, Vancouver (CA); Greg Alan WALTERS, Exton, PA (US); Philip J. HAARSTAD, Chanhassen, MN (US)

(73) Assignee: Excision Medical, Inc., Exton, PA (US)

(21) Appl. No.: 19/429,669

(22) Filed: Dec. 22, 2025

Related U.S. Application Data
(63) Continuation of application No. 17/782,238.
(60) Provisional application No. 62/944,109, filed on Dec. 5, 2019.
(60) Provisional application No. 63/022,119, filed on May 8, 2020.

(51) Int. Cl. A61M 25/00; A61B 17/00; A61B 17/221; A61B 17/32; A61B 17/3207; A61F 2/01

(52) U.S. Cl. CPC A61M 25/00; A61B 17/320016; A61B 17/32075; A61B 2017/00243; A61B 2017/2212; A61F 2/011

(57) ABSTRACT

The present disclosure is an assembly and related method that allows laceration of a valve leaflet.

### CROSS-REFERENCE TO RELATED APPLICATIONS

This application is a continuation of U.S. application Ser. No. 17/782,238, filed Jun. 3, 2022, which is a national phase application of PCT Application No. PCT/US2020/063504, filed Dec. 5, 2020, which claims the benefit of and priority to U.S. Provisional Application No. 63/022,119, filed May 8, 2020, and U.S. Provisional Application No. 62/944,109, filed Dec. 5, 2019. The entire contents of each application listed in this paragraph are incorporated by reference into the present application.

### TECHNICAL FIELD

The present disclosure relates to a catheter and in particular to a catheter assembly with a cutting element.

### BACKGROUND

Transcatheter aortic valve replacement (TAVR) is an alternative option for the treatment of patients with severe calcific aortic stenosis. Indeed, TAVR may become the preferred therapy for all patients irrespective of surgical risk. However, transcatheter heart valves (TVH) may fail in the future and repeat intervention may be required. So-called redo-transcatheter aortic valve implantation (TAVI) or TAVR may lead to risks of coronary obstruction due to the leaflet of the failed THV being pushed up by the new THV and leading to obstruction of blood flow to the coronary arteries.

TAVR in failed surgical bioprostheses is common. However, TAVR in failed transcatheter bioprostheses (i.e. transcatheter heart valve-in-transcatheter heart valve) will also become increasingly common. In both situations there is a risk of coronary obstruction. The risk of coronary obstruction can be predicted with the use of cardiac computed tomography. If the predicted risk of coronary occlusion is high, then percutaneous valve-in-valve intervention may be prohibitive. In some cases, the cause of the coronary obstruction is related to the leaflets of the failed surgical or transcatheter heart valve that are pushed up and prevent flow of blood to the coronary arteries. To overcome this challenge, one approach is implementing the bioprosthetic aortic scallop intentional laceration to prevent iatrogenic coronary artery obstruction procedure, or “BASILICA.” The BASILICA procedure uses catheter electrosurgery to split the offending bioprosthetic leaflet, creating a triangular space that facilitates blood flow to the coronary artery. The BASILICA technique relies on electrification of a wire. This technique is complex with prolonged procedure times, often taking several hours versus the 20-30 mins. for a standard transfemoral valve in valve procedure. The BASILICA technique also requires a number of steps in order to achieve successful splitting of the valve leaflet. Currently only a few specialized centers perform the BASILICA procedure and it is unlikely to become a routine due to its complexity.

### SUMMARY

There is a need for improved systems, devices and procedures for leaflet laceration in failed transcatheter heart valves. The present disclosure includes embodiment include a catheter assembly that allows for reproducible and safe cutting of the bioprosthetic transcatheter leaflet.

An embodiment of the disclosure may include a catheter assembly. The catheter assembly also includes an outer catheter having a proximal end, a distal end, a first channel that extends from the distal end toward the proximal end, and a port that opens to the first channel and is positioned toward the distal end. The catheter assembly also includes an inner catheter having a distal end, a proximal end, and a second channel that extends from the proximal end toward the distal end. The inner catheter may be sized and configured to slide within the first channel with the distal end configured to exit through an opening. The catheter assembly also includes a cutting assembly configured to move within the second channel. The cutting assembly has a cutting element that is configured to lacerate a leaflet. The cutting assembly is configured to transition from a retracted configuration, where the cutting element is contained within the second channel, into an extended configuration, where the cutting element extends out of the distal end of the inner catheter to facilitate laceration of a leaflet. A cutting assembly includes a cutting element configured to permit laceration of a leaflet, an even heavily calcified leaflet, with a less technically demanding method.

In an embodiment, the cutting assembly is configured to transition from a retracted configuration, where the cutting element is contained within the second channel, into an extended configuration, where the cutting element extends out of the distal end of the inner catheter to facilitate laceration of a leaflet. A cutting assembly includes a cutting element configured to permit laceration of a leaflet, an even heavily calcified leaflet, with a less technically demanding method.

In an embodiment, the catheter assembly may include at least one actuator coupled to the cutting element. The actuator is configured to cause the cutting assembly to transition from the retracted configuration into the extended configuration. The cutting element is not electrified.

In an embodiment, the cutting element has a shaft, a distal tip, and an angled leg that extends from and is angled relative to the shaft, the cutting element having a first sharp region defining along the tip and a second sharp region is defined where the shaft and the leg intersect, where the first sharp region is configured to pierce a leaflet and the second sharp region is configured to lacerate the leaflet. The cutting element is separate from the inner catheter but insertable through the second inner channel.

In another embodiment, the outer catheter further may include at least one marker configured to permit identification of a position of the distal end relative to the leaflet.

An embodiment of the disclosure may include a method for lacerating a leaflet of a heart valve. The method also includes inserting a catheter into an aorta. The method also includes anchoring the catheter to a frame of a heart valve positioned in the aorta. The method also includes advancing a cutting element from within a channel of the catheter toward a base of the leaflet in a distal direction until a distal sharp tip of the cutting element punctures the leaflet. The method also includes retracting the catheter in a proximal direction with the cutting element in the extended configuration to lacerate the leaflet. The method also includes retracting the cutting element into a channel of the catheter.

In an embodiment of the method, inserting that catheter into the aorta includes advancing the catheter over a guidewire. Advancing a cutting element of the catheter toward the base of the leaflet includes advancing the cutting element over a guidewire and through a port of the catheter.

In an embodiment of the method, advancing the cutting element may include advancing the cutting element through an inner catheter disposed in the catheter.

In an embodiment of the method, anchoring the catheter proximate to the frame of the heart valve further may include expanding at least one engagement member such that the engagement member engages the frame.

### BRIEF DESCRIPTION OF THE DRAWINGS

The foregoing summary, as well as the following detailed description, may be better understood when read in conjunction with the appended drawings. The drawings show illustrative embodiments of the disclosure. It should be understood, however, that the application is not limited to the precise arrangements and instrumentalities shown.

FIG. 1A is a schematic view along a superior-inferior direction of an aorta with an implanted heart valve therein;

FIG. 1B is a schematic sectional view of the heart valve and aorta shown in FIG. 1;

FIG. 2 is a schematic plan view of a catheter assembly according to an embodiment of the present disclosure;

FIG. 3 is a schematic plan view of the catheter assembly shown in FIG. 3, illustrating a cutting element projecting from a distal end 29 of a catheter;

FIG. 4 is a schematic plan view of a distal end 29 of the catheter shown in FIGS. 2 and 3;

FIG. 5 is a schematic plan view of an alternative embodiment of a distal end 29 of the catheter shown in FIGS. 2 and 3;

FIG. 6 is a schematic plan view of another alternative embodiment of a distal end 29 of the catheter shown in FIGS. 2 and 3;

FIG. 7 is a perspective view of a distal end 29 of the catheter shown in FIG. 3, with the cutting element projecting from the catheter;

FIG. 8 is a perspective view of the distal end 29 of the catheter shown in FIG. 7, illustrating the cutting element in a curved or pigtail configuration;

FIG. 9 is a perspective view of the distal end 29 of the catheter shown in FIG. 7, illustrating the cutting element in a curved configuration, according to another embodiment of the present disclosure;

FIG. 10A is a schematic plan view of another alternative embodiment of a distal end 29 of the catheter;

FIG. 10B is a schematic plan view of the distal end 29 of the catheter shown in FIG. 10A, illustrating the cutting element projecting from the catheter;

FIG. 10C is a detailed view of the cutting element shown in FIG. 10B;

FIGS. 11A and 11B illustrate an actuator used to advance the cutting element from an insertion configuration into an extended configuration, respectively;

FIG. 12 illustrates a distal end 29 of the catheter approaching a frame of an implanted valve adjacent to the leaflet;

FIG. 13 illustrates a distal end 29 of the catheter being deflected toward the base of the leaflet;

FIG. 14 illustrates a cutting element advance from the distal end 29 of the catheter and piercing the base of the valve leaflet;

FIG. 15 illustrates the cutting element transitioning into a curved configuration; and FIG. 16 illustrates the catheter and cutting element being retracted in a proximal direction to lacerate the leaflet;

FIG. 17 is a schematic plan view of a catheter assembly according to another embodiment of the present disclosure;

FIG. 18 illustrates a catheter assembly according to another embodiment of the present disclosure, positioned to lacerate the leaflet;

FIG. 19 illustrates a catheter assembly shown in FIG. 17, showing an engagement member of the outer catheter engaged with an inner surface of a frame of a heart valve;

FIG. 20 illustrates a catheter assembly shown in FIG. 17, showing an engagement member of the outer catheter engaged with an outer surface of a frame of a heart valve;

FIG. 21 illustrates a catheter assembly shown in FIG. 17, showing a pair of engagement members of the outer catheter engaged with an inner and outer surface of a frame of a heart valve;

FIG. 22 illustrates a catheter assembly shown in FIG. 17, showing an inner catheter exiting from a port of the outer catheter and a first engagement member engaged with the frame;

FIG. 23 illustrates a catheter assembly shown in FIG. 17, showing an inner catheter exiting from a port of the outer catheter and a second engagement member engaged with the frame;

FIG. 24 illustrates a catheter assembly shown in FIG. 17, showing an inner catheter exiting from a port of the outer catheter and a pair of engagement members engaged with the frame;

FIG. 25 illustrates a catheter assembly shown in FIG. 22, showing the cutting element extending from the inner catheter and the port of the outer catheter;

FIG. 26 illustrates a catheter assembly shown in FIG. 23, showing the cutting element extending from the inner catheter and the port of the outer catheter;

FIG. 27 illustrates a catheter assembly shown in FIG. 24, showing the cutting element extending from the inner catheter and the port of the outer catheter;

FIG. 28 illustrates a catheter assembly shown in FIG. 25 showing the cutting element extending from the inner catheter and the port of the outer catheter, and catheter assembly pulled in antegrade direction to lacerate the leaflet;

FIG. 29 is a side sectional view of a portion of the catheter assembly shown in FIG. 17, with the cutting element in a retracted configuration;

FIG. 30 is a side sectional view of a portion of the catheter assembly shown in FIG. 17, with the cutting element in adjacent the leaflet; and

FIG. 31 is a side sectional view of a portion of the catheter assembly shown in FIG. 17, with the cutting element in piercing the leaflet.

### DETAILED DESCRIPTION OF ILLUSTRATIVE EMBODIMENTS

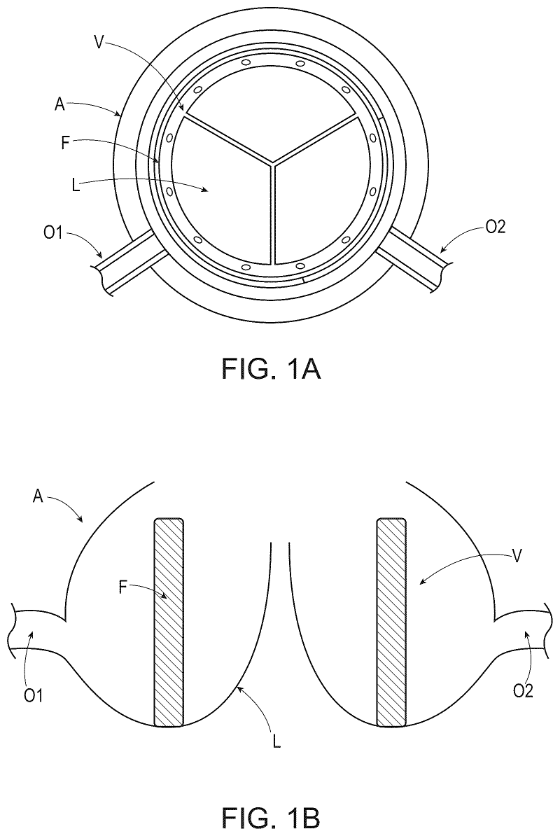

Embodiments of the present disclosure as described include a catheter assembly with a cutting element which is designed to cut surgical and transcatheter bioprosthetic valve leaflets. Such embodiment as described herein simplify the process of aortic leaflet laceration. In an embodiment, a catheter assembly may be used to achieve bioprosthetic valve laceration. As shown in FIGS. 1A and 1B, an implanted valve V may be implanted in an aorta A. The valve V may include a F and typically three leaflets L that form the valve structure. The valve V is typically positioned proximate a coronary ostia O1,O2. However, the frame F may or may not obstruct the coronary ostia O1,O2.

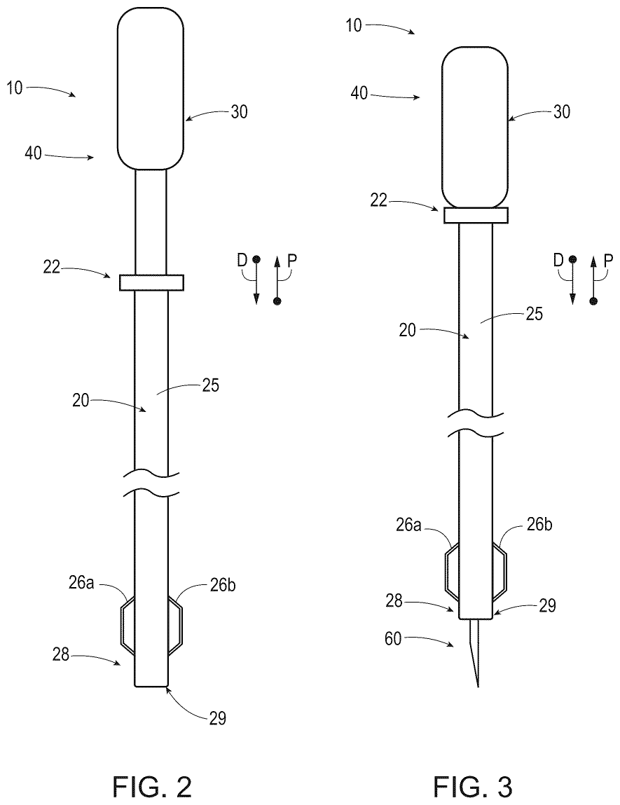

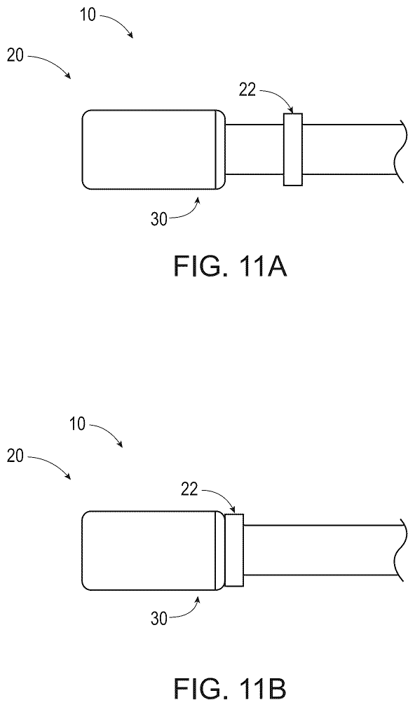

As shown in FIGS. 2-4, the catheter assembly 10 may include a catheter 20 and cutting assembly 40. One or more actuators (FIGS. 11A, 11B) may be included to control operation of the catheter assembly 10. The cutting assembly 40 may be part of the catheter 20 or it may be a separate component that is inserted through the catheter 20.

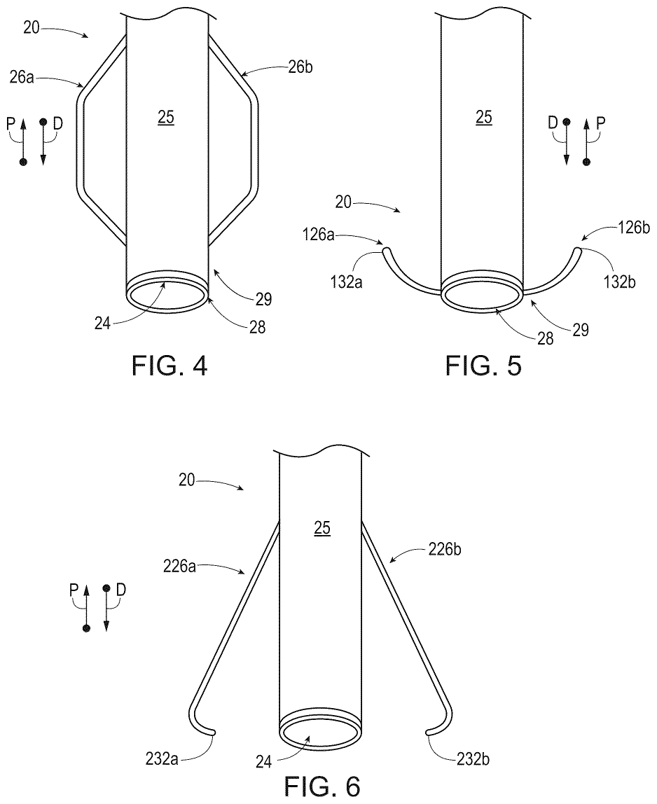

Continuing with FIGS. 2-4, the catheter 20 may include a hub 22 at its proximal end 23 and an elongated body 25 coupled to the hub 22. The elongated body 25 includes a shaft portion, a secondary curve, a primary curve, one or more radiopaque markers R, and a distal tip 28. The primary and secondary curves are not illustrated in the drawings. The distal tip 28 defines the distal end 29 of the catheter 20. As best shown in FIG. 4, the catheter 20 includes an inner channel 24 (FIG. 4) that extends from the proximal end 23 to the distal end 29 of the elongated body 25. The inner channel 24 is sized to contain or receive therethrough all or portions of the cutting assembly 40. The inner channel 24 is also sized to receive other surgical devices therethrough. For example, the catheter 20 can receive a guidewire such that an over-the-wire technique may be used. In cross-section, catheter 20 may include an inner liner, a middle reinforcing layer (e.g. a braid), and an outer layer or outer jacket. In another embodiment, the catheter 20 would also be able to accommodate different shaped inner catheters to achieve a suitable relationship of the distal catheter tip 28 to the leaflet. For example, this configuration may provide for functionality similar to the use of a 5F/6F 120 mm IM catheter inside an AL type catheter, i.e. a mother and daughter technique. The catheter 20 may be configured to transition in response to operator input to assume different degrees of flexion of the distal tip 28 to account for different patient anatomy.

The longitudinal shape of the catheter can vary as needed. For instance, the catheter 20 can have a shape according to the Amplatz Guide that includes, but is not limited to AL-1, AL-2, AL-3, AL-4, etc. Other common shapes are possible as well. In one example, the catheter may have an outer cross-sectional dimension sized for insertion into the aorta. For instance, the catheter may be either 7 French or 8 French, e.g. between 1.98 mm and 2.30 mm inner diameter. However, larger or smaller sized catheters may be used in certain instances. The catheter tip 28 may be deflectable or bendable as needed. The catheter 20 may also be configured to accommodate different shaped inner catheters.

Turning to FIGS. 4-6, the distal end 29 of the catheter 20 is configured to permit positioning of the distal tip 28 proximate to and in contact with the base B of the valve leaflet L (base and leaflet not shown). As shown in FIG. 4, the catheter 20 may include a plurality of positioning elements 26a, 26b that extend outwardly with respect to the elongated body 25 of the catheter 20. The positioning elements 26a, 26b may be flexible wires that are configured to expand outwardly upon actuation. Alternatively, the positioning elements 26a, 26b have a natural state that extends outwardly as shown in FIG. 4, and a compressed state whereby the positioning members are compressed against the outer surface of the catheter 20 (not shown). FIGS. 5 and 6 illustrate alternative embodiments of positioning elements. For example, FIG. 5 illustrates an alternative catheter 20 with curved positioning elements 126a, 126b that curve away from the elongated body 25. Terminal ends 132a, 132b of each element 126a, 126b are spaced away from the body 25 of the catheter 20 such that the concave sides of each positioning element face a proximal direction P. The proximal direction P is generally in a direction toward the user and a distal direction D is generally in a direction toward the distal end 29. Each curved positioning element 126a, 126b is configured to guide the catheter 20 in position in the aorta. In another example shown in FIG. 6, an alternative catheter 20 has angled positioning elements 226a, 226b that project away from elongated body 25 of catheter in angled orientation. Terminal ends 232a, 232b of each element 226a, 226b are spaced away from the distal tip 28 and guide the catheter 20 in position. In each embodiment described herein, the positioning elements 226a, 226b help orientate the catheter to the base of the leaflet. The positioning elements may also help allow anchoring and positioning of the catheter when leaflet puncture is being performed. Additionally, one embodiment would be configured to allow the catheter to orientate down to the base of the cusp so that the base of the leaflet can be lacerated. Furthermore, another embodiment would be deployable anchors that also would allow orientation to the base of the leaflet.

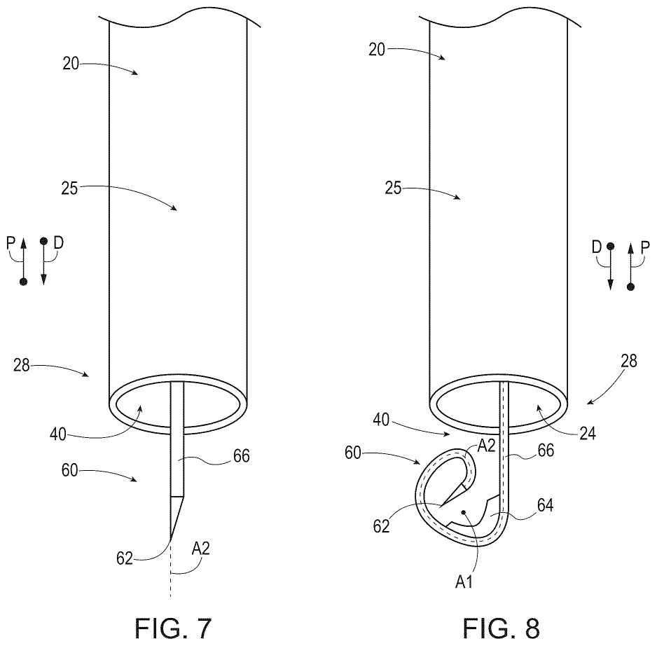

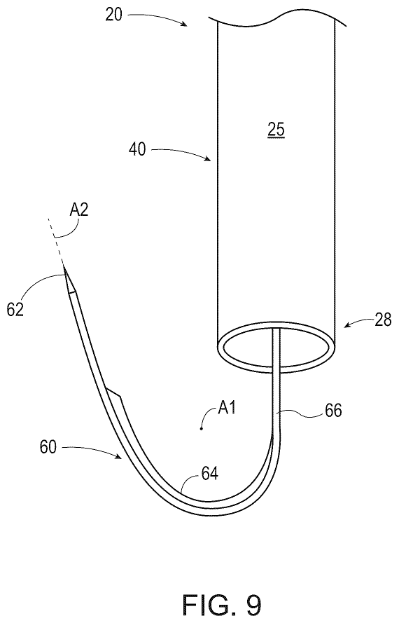

As shown in FIGS. 7 and 8, the cutting assembly 40 includes a cutting element 60 configured to lacerate the valve. The cutting element 60 include a sharp tip 62 and a cutting edge 64 that extends from the sharp tip 62 in a proximal direction P. The sharp tip 62 of the cutting element 60 is configured to puncture the base B of the bioprosthetic leaflet L. The cutting element 60 may also include a flexible cutting body 66 that defines the sharp tip 62 and the cutting edge 64. The flexible cutting body 66 is configured to transition into a curved shape, as shown in FIG. 8, to lacerate the valve leaflet. In one example, in the curved shape, the flexible cutting body 66 curves about an axis A2 that is perpendicular to an axis Al of the cutting element 60. In this manner, the curve configuration helps minimize damage to the valve or other tissue during laceration. In alternative embodiments, the cutting element may comprise multiple components to facilitate transition of the cutting element 60 into a curved shape during use. The curved configuration shown in FIG. 8 may be referred to as pigtail configuration. FIG. 9 illustrates cutting element 60 curved toward the proximal direction P. In FIG. 9, the distal tip 62 of the cutting element 60 is not curved toward itself, unlike that shown in FIG. 8.

In one example, the catheter 20 includes at least one actuator 30 (see also FIG. 11A an 11B) coupled to the cutting element 60. In such an example, the actuator 30 is configured to transition the cutting assembly 40 through: 1) a first actuation phase where the cutting assembly 40 transitions from a retracted configuration into an extended linear configuration wherein the cutting element 60 projects from the distal end 29 of the elongated body 25; 2) a second actuation phase where the flexible cutting body 25 transitions from the extended linear configuration (FIG. 7), where the distal tip 62 is positioned to puncture the valve leaflet, into the curved configuration, where the cutting edge 64 is curved into the curved shape (FIGS. 8 and 9); and 3) a third actuation phase where the cutting element 60 is configured to transition from the curved configuration back into the retracted configuration (FIG. 2). Accordingly, the cutting element 60 in the curved configuration allows the operator to pull the catheter in an antegrade direction relative to the flow of blood and permit the cutting edge 64 to lacerate the leaflet. When the laceration is complete, the cutting element 60 can transition back into the retracted configuration and the catheter 20 can be removed or other devices can be used to access the coronary artery as needed.

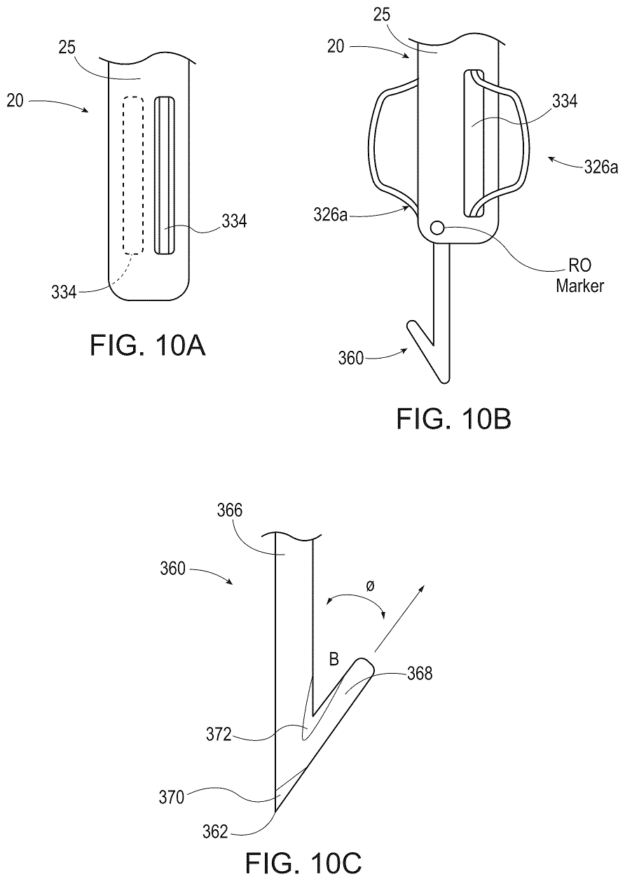

Turning to FIGS. 10A-10C, an alternative embodiment of a cutting element 360 is illustrated. In accordance with the illustrated embodiment, the catheter assembly 340 and/or the catheter 20 includes one or more positioning elements 326a, 326b which extend outward from the catheter 20, a cutting element 360, and one or more actuators 330 to control operation of the positioning elements 326a, 326b and the cutting element 360. The positioning elements 326a, 326b can extend out from elongated slots 334 in the catheter body 25 and are actuated by one or more actuators 330 proximate the handle (not shown). The positioning elements 326a, 326b may be formed from nitinol wires as needed. As shown in FIG. 10C, the cutting element 360 includes a shaft 366, a distal tip 362 or apex at the end of the shaft 366, and an angled leg 368 that extends generally in an angular direction relative to the shaft 366. The leg 368 and shaft 366 define an angle φ. Angle φ may be range between 30 degrees and 60 degrees. In one example, angle φ is about 45 degrees. The cutting element 360 may include a first sharped region 370 defined at the apex 362 and a second sharpened region 372 is defined inside the apex where the shaft 366 and leg 368 meet. The second sharpened region 372 is configured to lacerate the leaflet when the catheter assembly 340 is pulled antegrade relative to the flow of blood. In some instances, antegrade relative to the flow of blood may be the same as a proximal direction P (relative to the instrument). The cutting element 360 may be referred to as a cutting blade with a sharp outer region and inner region. In addition, the cutting element 360 may include a guard element that covers the first outer region to prevent any further damage to surrounding structures. In another embodiment, the cutting element would be able to be delivered through any standard 7F guiding catheter, e.g. Pachyderm.

In any particular embodiment of the catheter assembly described herein, the cutting elements and the catheter can be locked together, so that when the leaflet is punctured, the relationship between the catheter and cutting element can be maintained and the operator can pull both to achieve laceration of the leaflet. In such an example, the catheter and/or cutting assembly include a locking element that locks the components together as needed. The locking element may be activated or deactivated as needed.

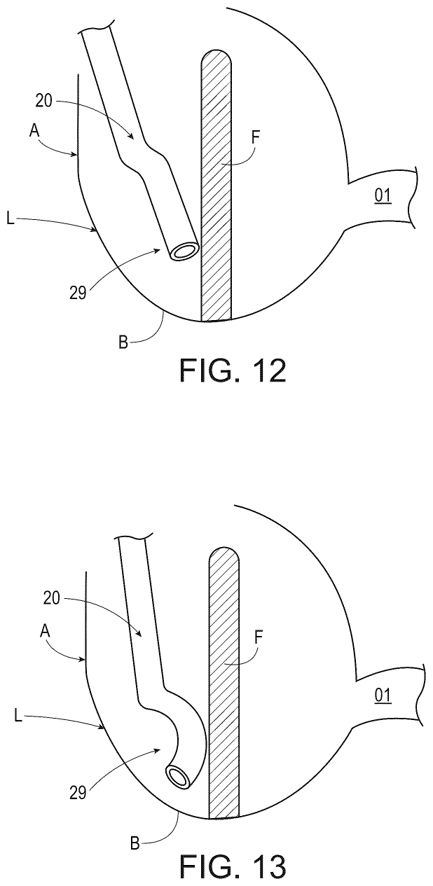

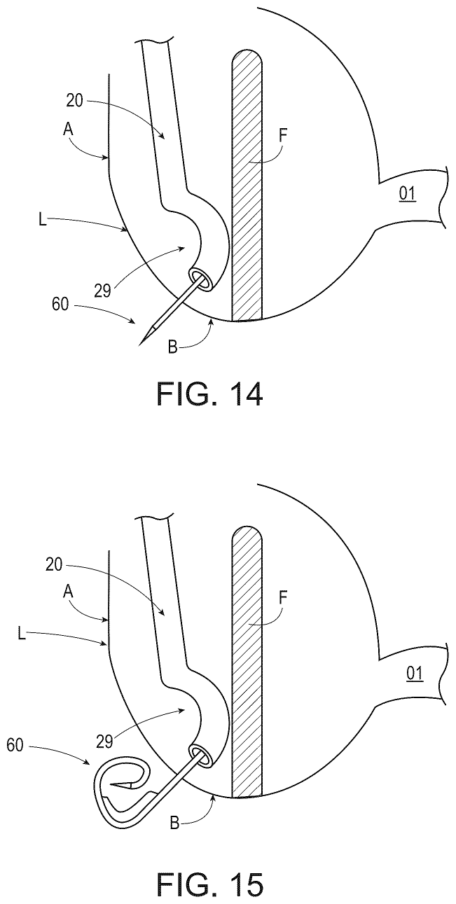

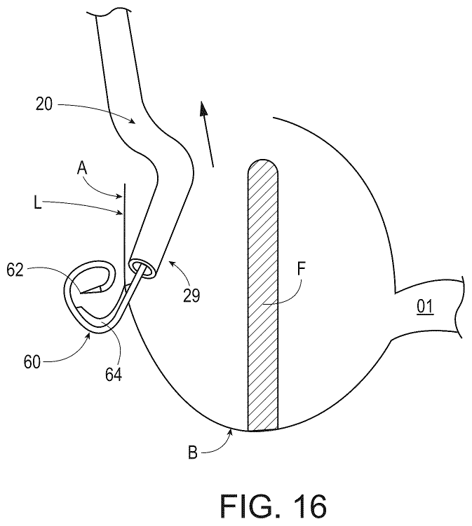

Referring to FIGS. 12-16, in use, the catheter may be inserted trans-femoral or radially and advanced so that its distal end 29 approaches the aorta A. More specifically, the distal end 29 of the catheter 20 is advanced until the distal end 29 is positioned at the base B of the surgical valve leaflets L. The positioning of the distal end 29 of the catheter 20 may be accomplished using standard techniques and fluoroscopic imaging and/or transesophageal imaging. Once the catheter 20 is positioned in the base of the bioprosthetic leaflet, the cutting element 60 may be deployed. Many different cutting elements and configurations may be used, as explained herein. For example, any cutting element described herein and shown in FIGS. 12-16 may be used. For ease of illustration, only the cutting element is shown in FIGS. 12-16 which illustrate the method of use. The use of the catheter assembly is not limited strictly to what is shown in FIGS. 12-16.

Continuing with FIGS. 12-16, the cutting element 60, and in particular its sharp tip 62, may be advanced out of the distal end 29 of the catheter 20 and can be used to puncture through the bioprosthetic valve leaflet. Once the cutting element 60 has perforated through the leaflet, the cutting element 60 can transition into a curved shape, e.g. a pigtail, to prevent damage to the surrounding structure. The inner portion of the cutting element, for instance the cutting edge 64, is configured and positioned to lacerate the leaflet. The cutting edge 64 and pigtail tip 66 of the cutting element 60 may be locked into position for laceration of the leaflet. When the catheter 20 is retracted by the operator, the leaflet may be lacerated from the base to the free edge by the cutting element 60, ideally in the middle of the bioprosthetic leaflet creating two equal remaining portions. Once laceration is completed, the cutting element 60 can be withdrawn back into the catheter 20 and the catheter 20 can be removed from the body 25.

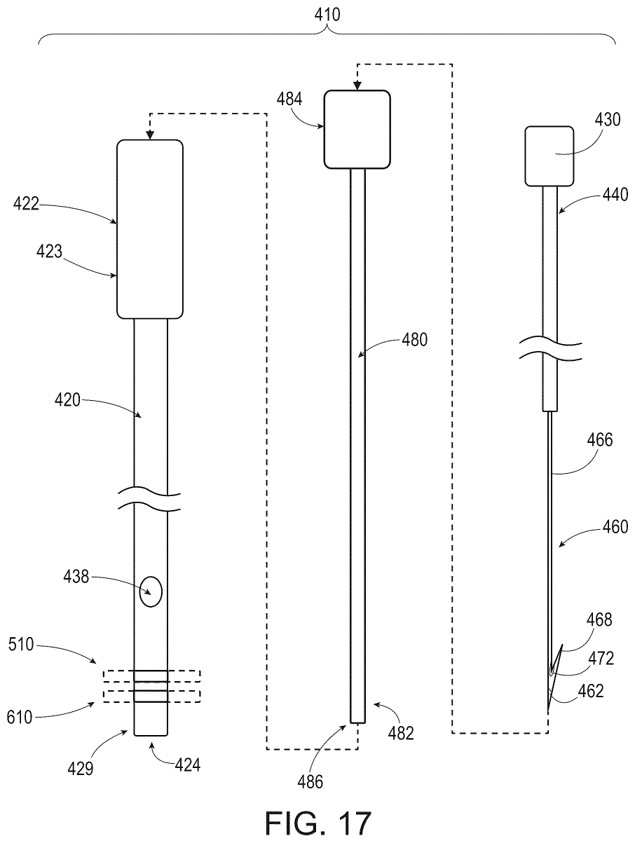

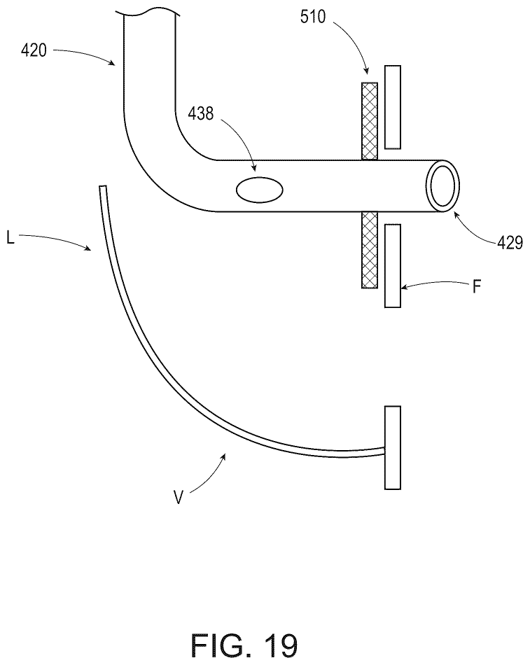

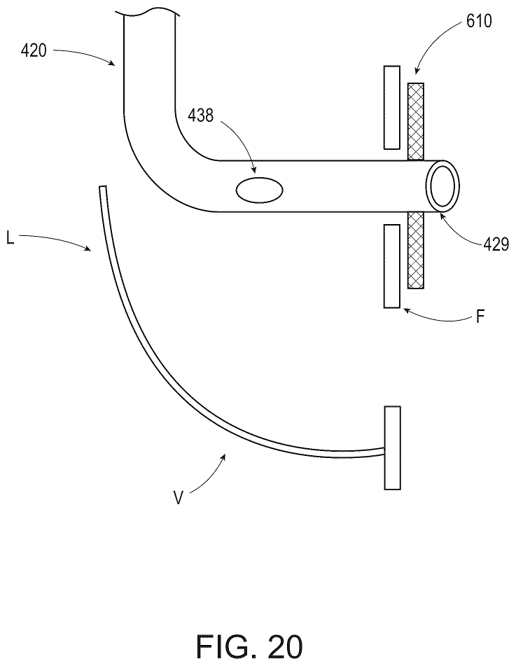

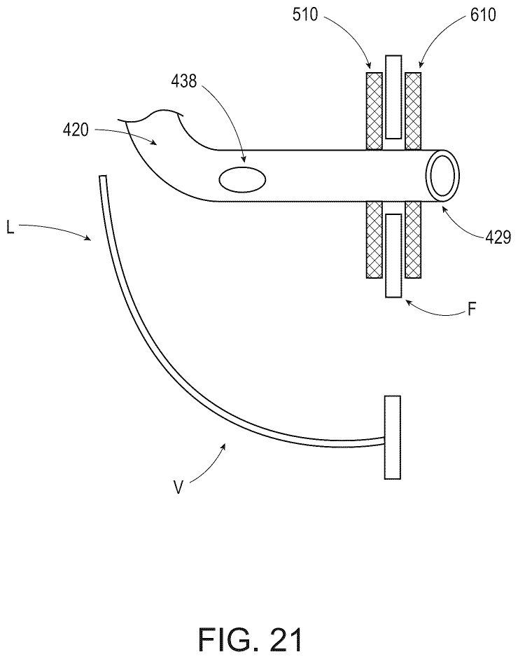

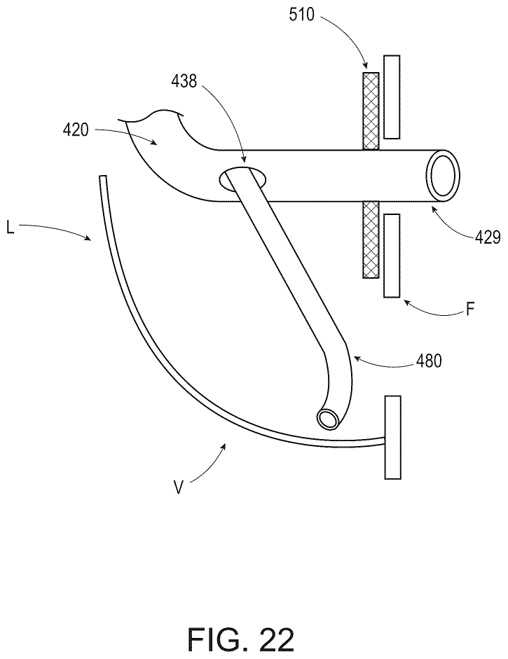

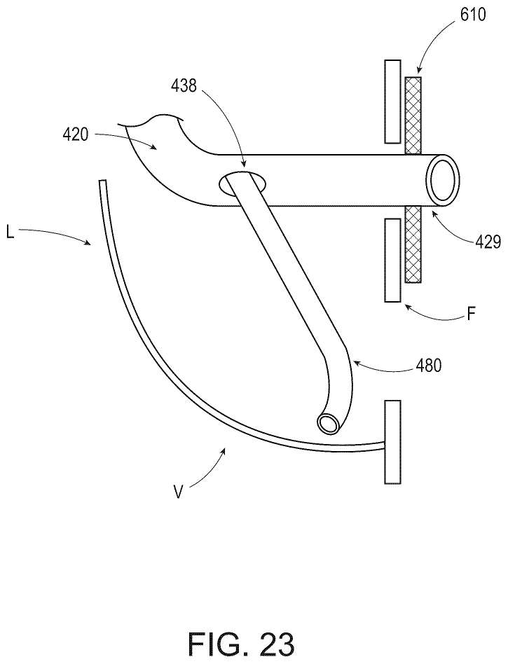

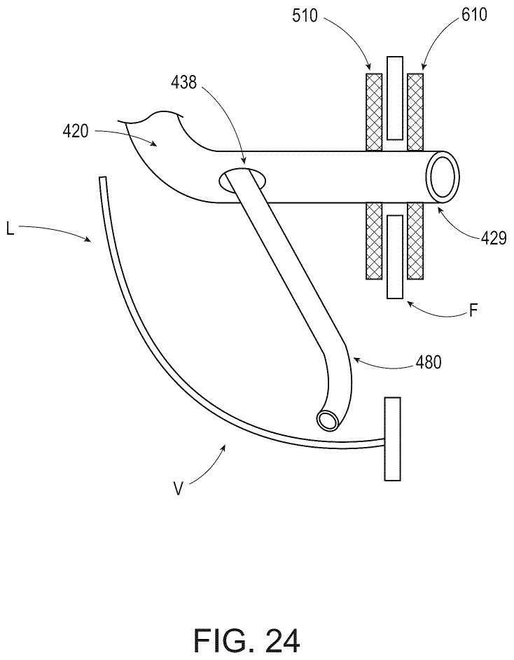

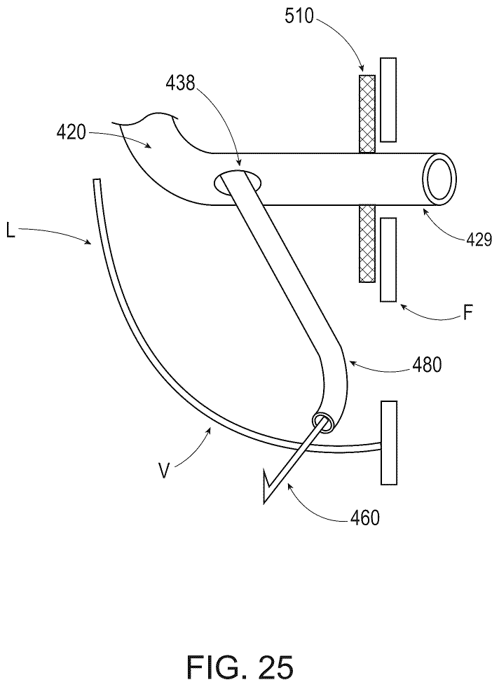

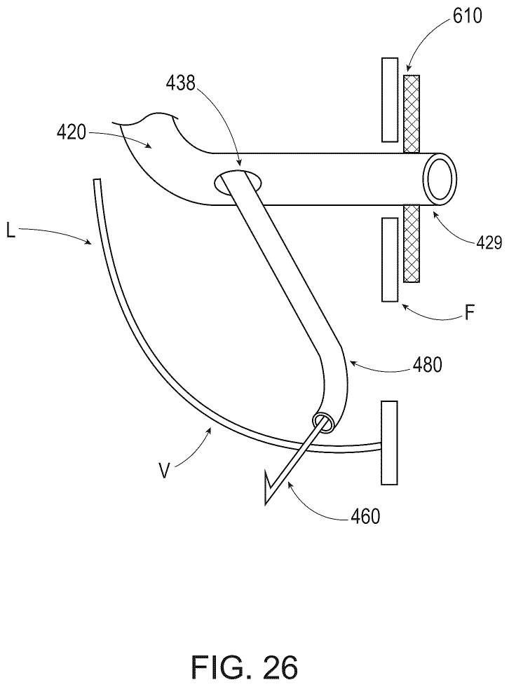

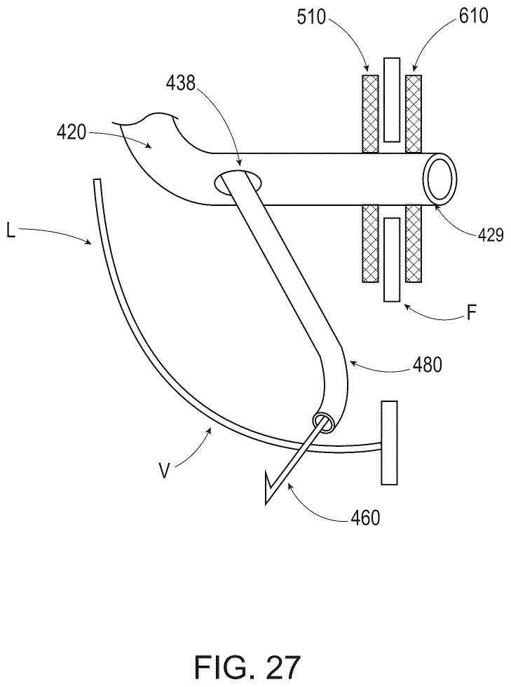

An alternative embodiment of a catheter assembly 40 is illustrated in FIGS. 17-31. As shown in FIGS. 17 and 29-31, a catheter assembly 410 that is configured to lacerate (cut) the leaflet L of a transcatheter heart valve V in a cardiovascular system. The catheter assembly 410 also includes an outer catheter 420 having a proximal end 422, a distal end 429, a first channel 424 that extends from the distal end 429 toward the proximal end 422, and a port 438 that opens to the first channel 424 and is positioned toward the distal end 429. The outer catheter 420 may be referred to as an outer tube or outer tubular member. The outer catheter 420 further may include at least one marker (not shown) configured to permit identification of a position of the distal end relative to the leaflet.

The catheter assembly 410 also includes an inner catheter 480 having a distal end 482, a proximal end 484, and a second channel 486 (not shown) that extends from the proximal end 484 toward the distal end 482. The inner catheter 480 may be referred to as an inner tube or inner tubular member. The inner catheter 480 may be sized and configured to slide within the first channel 428 with the distal end 482 configured to exit through the port 438. In an embodiment, the inner catheter 480 would have a deflectable distal end 482 that would be controlled from the proximal end 422 of the catheter assembly 410. The inner catheter 480 will be able to deflect into different shapes to allow passage through the cell of a transcatheter heart valve. The inner catheter 480 may have different shapes to allow its optimal position proximate the base of the transcatheter heart valve leaflet.

The catheter assembly 410 also includes a cutting assembly 440 configured move within the second channel 486 (not shown). The cutting assembly 440 includes the cutting element 460 described herein. More specifically, the cutting assembly 440 is configured to transition from a retracted configuration, where the cutting element 460 is contained within the second channel 486, into an extended configuration, where the cutting element 460 extends out of the distal end of the inner catheter 480 to facilitate laceration of a leaflet.

In an embodiment, the catheter assembly 440 may include at least one actuator 430 coupled to the cutting element 460. The actuator 430 is configured to cause the cutting assembly 440 to transition from the retracted configuration into the extended configuration. In one example, the cutting element 460 is not electrified. In an alternative example, however, the cutting element can be electrified.

In an embodiment, the cutting element 460 has a shaft 466, a distal tip 462, and an angled leg 468 that extends from and is angled relative to the shaft 466. The cutting element 460 has a first sharp region 470 defined along the tip 462 and a second sharp region 472 is defined where the shaft 466 and the leg 468 intersect. The first sharp region 470 is configured to pierce a leaflet and the second sharp region 472 is configured to lacerate the leaflet. The cutting element 460 is separate from the inner catheter but insertable through the second inner channel 486.

In an embodiment, the catheter assembly 440 may include an engagement assembly. The engagement assembly may include at least one engagement member 510, 610 configured to couple the outer catheter 420 to a frame F of a heart valve V. As illustrated, the engagement assembly has a first engagement member 510 and a second engagement member 610 each configured to couple to the frame of the heart valve. The engagement members 510, 610 may be formed from nitinol. The engagement members 510, 610 may be coupled to the outer catheter at or near its distal end 29. The engagement members 510, 610 are configured to transition from an insertion configuration, which is generally a collapsed shape, into the engagement configuration, which has an expanded shaped. In the engagement configuration, the engagement members 510, 610 are deployed into a generally circular shape along a plane that is perpendicular to an axis of the outer catheter. The engagement members 510, 610 can be retracted or collapsed when within the outer catheter. When the engagement members 510, 610 are deployed, they transition into a trumpet like shape that has an inner channel through which various devices can pass through.

The engagement members 510, 610 may have a differential distribution allowing more layers of material forming the member at the distal edges. This mass distribution described herein allows for differential strength at the engagement members. In an embodiment the engagement members 510, 610 may be composed of nitinol. In another embodiment the engagement members 510, 610 may be a laser cut metal that will be able to be deployed at the distal edge of the outer catheter.

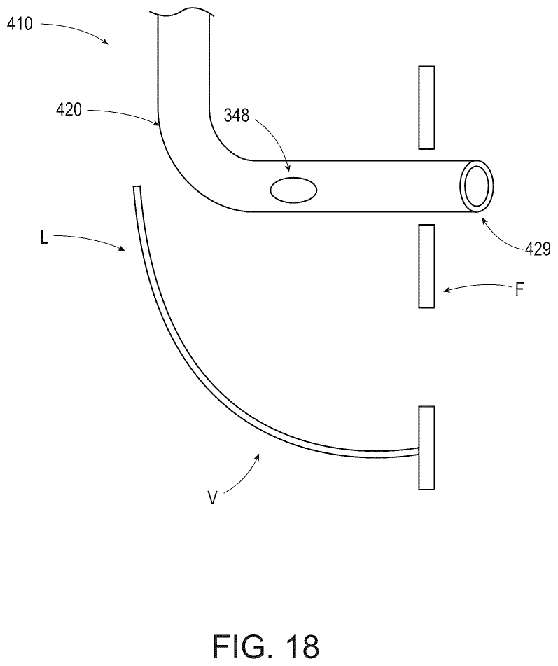

In an embodiment, a catheter assembly 410 may be used to lacerate a leaflet of a heart valve, as shown in FIGS. 18-31. In an exemplary method, the catheter assembly 410 is advanced to the aortic root and positioned adjacent to the transcatheter heart valve frame above the desired leaflet L. In on example, the engagement member 610 is deployed just proximal to the distal end 429 of the catheter and adjacent to the inner part of the transcatheter heart valve frame. This will allow the operator to push forward and use the frame as support when advancing the inner catheter 480. An inner catheter 480 will be advanced from a port 438 of the outer catheter 420. When the inner catheter 480 is at the desired position, the cutting element 460 is advanced from a retraction configuration into an extended configuration in order to puncture through the leaflet L. Once the leaflet L is punctured the operator will then pull the entire catheter assembly in a proximal direction to lacerate the leaflet.

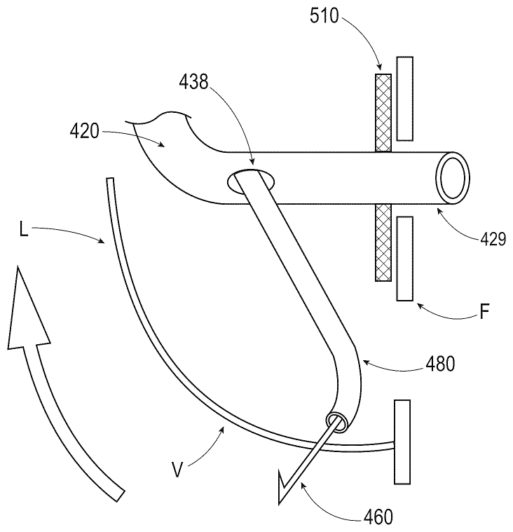

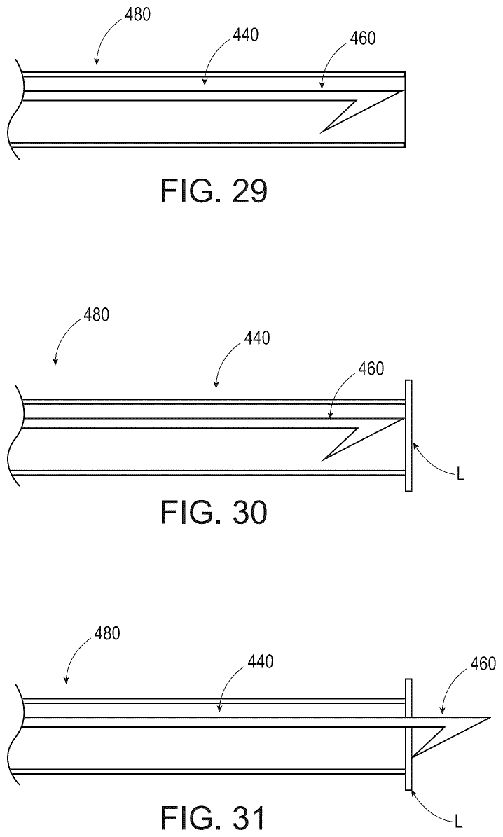

In FIGS. 29-31, it can be seen that initially, the cutting element 460 is generally disposed within the inner catheter 480 in the retracted configuration but is configured to move independently from and relative to the inner catheter 480. For instance, the operator will engage the surface of a leaflet with the inner catheter 480. The inner catheter 480 may be spring loaded so that it will move independent of the cutting element 460 as the inner catheter 480 pushes forward against the leaflet. As the operator pushes forward, the cutting element 460 can pierce the leaflet, as shown in FIG. 31. The inner catheter 480, however, will remain in the same position as the cutting element 460 advances through the leaflet L. At this point, the user can retract the assembly to lacerate the leaflet as described above.

While the disclosure is described herein, using a limited number of embodiments, these specific embodiments are not intended to limit the scope of the disclosure as otherwise described and claimed herein. The precise arrangement of various elements and order of the steps of articles and methods described herein are not to be considered limiting. For instance, although the steps of the methods are described with reference to sequential series of reference signs and progression of the blocks in the figures, the method can be implemented in an order as desired.

1. A catheter assembly, comprising:
a catheter having a proximal end, a distal end, and an inner channel that extends from the proximal end to the distal end; and
a cutting assembly having a cutting element, the cutting element having a sharp tip and a cutting portion that extends relative to the sharp tip in a proximal direction away from the sharp tip, wherein the cutting assembly is configured to transition from a retracted configuration, where the cutting element is contained within the channel, into a curved configuration, where the cutting element extends out of the distal end of the catheter into a curved shape to facilitate laceration of a leaflet.

2. The catheter assembly according to claim 1, wherein the cutting element includes a flexible cutting body that defines the sharp tip and the cutting portion, wherein the flexible cutting body is configured to transition into the curved shape.

3. The catheter assembly according to claim 1, wherein the flexible cutting body in the curved shape curves about an axis that is perpendicular to an axis of the cutting element.

4. The catheter assembly according to claim 1, wherein the cutting portion extends relative to and along a portion of the flexible cutting body.

5. The catheter assembly according to claim 2, wherein the flexible cutting body includes a blunt edge opposite the cutting portion.

6. The catheter assembly according to claim 2, wherein the flexible cutting body includes a first component that defines the sharp tip and a second component that defines the cutting portion and is coupled to the sharp tip, wherein the sharp tip is configured to curve upon application of a tensile force on the second component to cause the cutting element to transition into the curved configuration.

7. The catheter assembly according to claim 1, further comprising at least one actuator coupled to the cutting element, wherein the at least one actuator is configured to cause the cutting element to transition from the retracted configuration into the curved configuration.

8. The catheter assembly according to claim 1, wherein the actuator is configured to cause the cutting element to transition through:
a first actuation phase where the cutting element transitions from the retracted configuration into an extended linear configuration where the cutting element extends out from the distal end of the catheter;
a second actuation phase where the cutting element transitions from the extended linear configuration, where the sharp tip is positioned to puncture the leaflet, into the curved configuration, where the cutting element is curved into the curved shape; and
a third actuation phase where the cutting element transitions from the curved configuration into the retracted configuration.

9. The catheter assembly according to claim 1, wherein the cutting element is not electrified.

10. The catheter assembly according to claim 1, wherein the elongate tubular member further comprises at least one marker configured to permit identification of a position of the distal end relative to the leaflet.

11. A method for lacerating a leaflet of a heart valve, comprising:
inserting a catheter into an aorta;
advancing a cutting element from within a channel of the catheter toward a base of the leaflet in a distal direction until a distal sharp tip of the cutting element punctures the leaflet;
after the distal sharp tip punctures the leaflet, causing a portion of the cutting element that extends through the leaflet to transition into a curved configuration;
retracting the catheter in a proximal direction with the cutting element in the curved configuration to lacerate the leaflet; and
retracting the cutting element into a channel of the catheter.

12. The method according to claim 11, wherein inserting the catheter into the aorta includes advancing the catheter over a guidewire.

13. The method according to claim 11, wherein advancing the cutting element further comprises advancing the cutting element through an inner channel of the catheter.

14. The method according to claim 11, further comprising positioning a distal end of the catheter proximate the base of the leaflet.

15. The method according to claim 11, further comprising deflecting a distal end of the catheter toward the base of the leaflet.

16. The method according to claim 11, further comprising:
positioning a pre-shaped guide in the aorta adjacent to the base of the leaflet; and
causing the pre-shaped guide to deflect the distal end of the catheter toward the base of the leaflet.

17. A catheter assembly, comprising:
an outer catheter having a proximal end, a distal end, a first channel that extends from the distal end toward the proximal end, and a port that opens to the first channel;
an inner catheter having a second channel, the inner catheter being sized and configured to slide within the first channel with the proximal end configured to exit through the port; and
a cutting assembly disposed in the second channel, wherein the cutting assembly is configured to lacerate a leaflet.

18. The catheter assembly according to claim 17, wherein the cutting assembly includes a cutting element.

19. The catheter assembly according to claim 17, wherein the cutting assembly is configured to transition from a retracted configuration, where the cutting element is contained within the second channel, into an extended configuration, where the cutting element extends out of the second channel to facilitate laceration of a leaflet.

20. The catheter assembly according to claim 19, further comprising at least one actuator that is configured to cause the cutting assembly to transition from the retracted configuration into the extended configuration.

21. The catheter assembly according to claim 18, wherein the cutting element is not electrified.

22. The catheter assembly according to claim 18, wherein the cutting element is electrified.

23. The catheter assembly according to claim 18, wherein the outer catheter further comprises at least one marker configured to permit identification of a position of the distal end relative to the leaflet.

24. The catheter assembly according to claim 18 wherein the cutting element has a shaft, a distal tip, and a leg that extends from and is angled relative to the shaft when in an extended configuration.

25. The catheter assembly according to claim 24, wherein the cutting element has a first sharpened region defined by the tip and the second sharpened region is defined where the shaft and the leg intersect.

26. The catheter assembly according to claim 18, wherein the cutting element is separate from the inner catheter but insertable through the second inner channel.

27. A surgical assembly, comprising:
an elongated body having a proximal end, a distal end, and an inner channel that extends from the distal end toward the proximal end in a proximal direction;
a flexible elongated body carried in the inner channel and having a distal end; and
a cutting element carried by the flexible elongated body, the cutting element having a distal cutting tip at the distal end of the flexible elongated body, and a cutting portion that is positioned relative to the distal cutting tip in the proximal direction,
wherein the cutting element is configured to transition from a retracted configuration, where the cutting element is contained within the inner channel, into a cutting configuration, where the distal cutting tip extends out of the distal end of the elongated body to pierce a leaflet, and the cutting portion is positioned toward the proximal direction to facilitate laceration of a leaflet.

28. The surgical assembly according to claim 27, wherein the flexible elongated body is configured to transition into a curved shape.

29. The surgical assembly according to claim 28, wherein the flexible elongated body in the curved shape curves about an axis that is perpendicular to an axis of the cutting element.

30. The surgical assembly according to claim 28, wherein the cutting portion extends along a portion of the flexible elongated body.

31. The surgical assembly according to claim 30, wherein the flexible elongated body includes a first component associated with the distal cutting tip and second component associated with the cutting portion, where application of a tensile force on the second component causes the cutting element to transition into the cutting configuration.

32. The surgical assembly according to claim 28, further comprising at least one actuator coupled to the cutting element, wherein the at least one actuator is configured to cause the cutting element to transition from the retracted configuration into the cutting configuration.

33. The surgical assembly according to claim 29, wherein the at least one actuator is configured to cause the cutting element to transition through:
a first actuation phase where the cutting element transitions from the retracted configuration into an extended linear configuration where the cutting element extends out from the distal end of the elongated body;
a second actuation phase where the cutting element transitions from the extended linear configuration, where the distal cutting tip is positioned to puncture the leaflet, into the cutting configuration, where the cutting element is positioned toward the proximal direction to facilitate laceration of a leaflet; and
a third actuation phase where the cutting element transitions from the cutting configuration into the retracted configuration.

34. The surgical assembly according to claim 28, wherein the cutting element is electrified.

35. The surgical assembly according to claim 28, wherein the cutting element is not electrified.

36. The surgical assembly according to claim 28, wherein the cutting portion is a cutting inner portion.

37. A surgical assembly for insertion at or near a valve in a heart, the assembly comprising:
an elongated body having a proximal end, a distal end, and an inner channel that extends from the distal end toward the proximal end in a proximal direction; and
a cutting assembly movable in the inner channel, the cutting assembly having a flexible elongated body with a distal end, and a cutting element having a distal cutting tip at the distal end of the flexible elongated body, and a cutting portion that is positioned relative to the distal tip in a proximal direction away from the distal cutting tip,
wherein the cutting assembly is configured to transition from a retracted configuration, where the cutting element is contained within the channel, into a cutting configuration, where the distal cutting tip extends out of the distal end of the elongated body and the cutting portion is positioned toward the proximal direction to facilitate laceration of a leaflet of a valve when the distal end of the elongated body is positioned at or near the leaflet.

38. The surgical assembly according to claim 37, wherein the flexible elongated body is configured to transition into a curved shape.

39. The surgical assembly according to claim 38, wherein the flexible elongated body in the curved shape curves about an axis that is perpendicular to an axis of the cutting element.

40. The surgical assembly according to claim 38, wherein the cutting portion extends along a portion of the flexible elongated body.

41. The surgical assembly according to claim 37, wherein the flexible elongated body includes a first component associated with the distal cutting tip and a second component associated with the cutting portion, where application of a tensile force on the second component causes the cutting element to transition into the cutting configuration.

42. The surgical assembly according to claim 37, further comprising at least one actuator coupled to the cutting element, wherein the at least one actuator is configured to cause the cutting element to transition from the retracted configuration into the cutting configuration.

43. The surgical assembly according to claim 37, wherein the at least one actuator is configured to cause the cutting element to transition through:
a first actuation phase where the cutting element transitions from the retracted configuration into an extended linear configuration where the cutting element extends out from the distal end of the elongated;
a second actuation phase where the cutting element transitions from the extended linear configuration, where the distal cutting tip is positioned to puncture the leaflet, into the cutting configuration, where the cutting element is positioned toward the proximal direction to facilitate laceration of a leaflet; and
a third actuation phase where the cutting element transitions from the cutting configuration into the retracted configuration.

44. The surgical assembly according to claim 37, wherein the cutting element is electrified.

45. The surgical assembly according to claim 37, further comprising at least one positioning element configured to position the cutting element toward the leaflet.

46. The surgical assembly according to claim 37, wherein the cutting portion is a cutting inner portion.

47. A surgical assembly for insertion at or near a valve in a heart, the assembly comprising:
an outer elongated body having a proximal end, a distal end spaced from the proximal end, and a first channel that extends from the distal end toward the proximal end in a proximal direction;
an inner elongated body having a proximal end, a distal end spaced from the proximal end of the inner elongated body, and a second channel that extends from the distal end of the inner elongated body toward the proximal end of the inner elongated body in the proximal direction, where in the inner elongated body is configured to move within the first channel with the proximal end of the inner elongated body configured to exit the first channel; and
a cutting element carried by and being movable relative to the second elongated body, the cutting element having a distal tip configured to puncture and a leaflet and a curved portion configured to lacerate a leaflet of the valve,
wherein the cutting element is configured to transition from a retracted configuration, where the cutting element is contained within the second channel, into an extended configuration, where the curved portion of the cutting element extends out of the distal end of the inner elongated body to facilitate laceration of the leaflet.

48. The surgical assembly of claim 47, further comprising at least one actuator coupled to the cutting element, the at least one actuator configured to cause the cutting element to transition from the retracted configuration into the extended configuration.

49. The surgical assembly of claim 47, wherein the cutting element is configured to be electrified.

50. The surgical assembly of claim 47, wherein the cutting element is configured to not be electrified.

51. The surgical assembly of claim 47, wherein the cutting element has a shaft, a first cutting region defined by the distal tip, and a second cutting region spaced relative to the first cutting region in the proximal direction.

52. The surgical assembly of claim 47, wherein the outer elongated body includes at least one marker to permit identification of a position of the distal end.
